(19) United States
(12) Patent Application Publication
(10) Pub. No.: US 2010/0168225 A1
(43) Pub. Date: Jul. 1, 2010

(54) Small-Molecule Hepatitis C Virus (HCV) NS3/4A Serine Protease Inhibitors

(76) Inventors: Francois Jean, Vancouver (CA); Raymond Andersen, Vancouver (CA)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/594,790

(22) PCT Filed: Apr. 18, 2008
(86) PCT No.: PCT/CA2008/000737

§ 371 Date: Mar. 17, 2010

Related U.S. Application Data
(60) Provisional application No. 60/907,830, filed on Apr. 18, 2007.

(51) Int. Cl. A61K 31/37; C07D 311/20; A61P 31/14

(52) U.S. Cl. 514/457; 549/285; 549/289; 549/287; 549/288

(57) ABSTRACT

Compounds of Formula 5 and prodrug compounds of Formula 5 wherein G¹ is H or OH; G² is H, R′, or CO₂R′; G³, G⁴ and G⁵, are each independently selected from the group consisting of: H, R′, OH, OR′, F, Cl, Br, I, NH₂, NHR′,NR′₂, CN, SH, SR′, SO₃H, SO₃R′, SO₂R′, OSO₃R′, and NO₂; and R′ is a one to four carbon alkyl group are provided. Uses of these compounds and methods of medical treatment involving these compounds for the treatment of hepatitis C are also provided.

### TECHNICAL FIELD

The invention relates to compounds for use in hepatitis C therapy. More specifically to inhibitors of hepatitis C virus (NS)3/NS4A heterocomplex serine protease and the use of these compounds as therapeutics.

### BACKGROUND

Hepatitis C virus (HCV) infection has reached epidemic proportions worldwide, with >3% of the world population infected and 3-4 million people newly infected each year.

Non-structural (NS)3 serine protease plays an essential role in the Flaviviridae life cycle by mediating the maturational cleavage of the viral polyprotein precursor into functional proteins (replicase). The Flaviviridae NS3 enzyme and its viral cofactor (Hepatitis C virus (HCV) NS4A) form a membrane-associated non-covalent complex, and this association is strictly required for full NS3 protease activity and specificity. The recent discovery that HCV NS3/NS4A serine protease heterocomplex activity interferes with pathways of the innate immune response in addition to its essential roles in HCV replication has fostered the search for NS3 enzyme inhibitors and underlined the multifunctional nature of these NS heterocomplex enzymes. However, the enzymes' unusual induced-fit behavior and peculiar molecular architecture have presented considerable obstacles to the development of small-molecule inhibitors.

Currently, one of the most promising approaches to antiviral therapy is the development of inhibitors of the multifunctional non-structural (NS)3/NS4A heterocomplex serine protease, which is indispensable for HCV infectivity in the chimpanzee model. The inventors of this technology have reported a continuous in vitro high-throughput (HT) FRET-based protease assay for monitoring HCV NS3/4A enzymatic activity, which has been applied to identifying protein-based inhibitors (Richer M. et al (2004) JBC 279: 10222-10227). A specific and sensitive NS3/NS4A protease assay using a unique internally quenched fluorogenic substrate (IQFS) (Hamill P. and Jean F. (2005) Biochemistry 44:6586-96) has been developed. IQFS enables, for the first time, the direct, specific detection of NS3/NS4A protease activity within membrane fractions isolated from human cells expressing NS3/NS4A. This high-throughput fluorescence assay is an important tool for study of the properties of the HCV NS3/NS4A serine protease heterocomplex (Kuang, W. F., Lin, Y. C., Jean, R, et al. (2004) Biochem. Biophys. Res. Commun. 317: 211-217).

Coumarins are widely distributed in the plant kingdom and together with their derivatives they possess a range of biological activities.

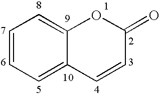

The structure of coumarin, including position numbering convention

WO2005/068450 describes 4-thiocoumarin compounds for the treatment of HCV. WO2005/068480 describes compounds containing a thio-aryl functionality at the 4 position of the general coumarin skeleton.

Two publications from Nippon Chemipher Co. Ltd. (Okamoto et al. (2001) Japan J. Pharmacology 87: 177-180; Okamoto et al. (2005) Current. Med. Chem—Anti Cancer Agents 5: 47-51) suggest using the coumarin osthole to prevent aviral hepatocellular carcinoma in mouse models.

### SUMMARY OF THE INVENTION

This invention is based, in part, on the discovery of the sensitivity of hepatitis C virus (NS)3/NS4A heterocomplex serine protease to particular substitutions to coumarin. This invention is also based, in part, on the discovery of the particular activity of specific substituted coumarins for hepatitis C virus (NS)3/NS4A heterocomplex serine protease as compared to other virus' serine proteases.

In one aspect of the present invention, there is provided a prodrug compound of Formula 5:

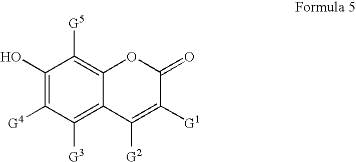

wherein G¹ is is H, R′, or CO₂R′; G³, G⁴ and G⁵, are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂; each R′ is independently a one to four carbon alkyl group; and from 1 to 5 OH moieties are replaced with a prodrug moiety.

In one aspect of the present invention, there is provided a prodrug compound described above wherein G¹ is OH or a prodrug moiety.

In one aspect of the present invention, there is provided a prodrug compound described above wherein G¹ is OH and not a prodrug moiety.

In one aspect of the present invention, there is provided a prodrug compound described above wherein each R′ is independently selected from the group consisting of: methyl and ethyl.

In one aspect of the present invention, there is provided a prodrug compound described above wherein G² is selected from the group consisting of: H, Me, Et, CO₂Me, and CO₂Et.

In one aspect of the present invention, there is provided a prodrug compound described above wherein G³ is H.

In one aspect of the present invention, there is provided a prodrug compound described above wherein G⁴ and G⁵ are independently H, OH or a prodrug moiety and at least one of G⁴ and G⁵ is OH or a prodrug moiety.

In one aspect of the present invention, there is provided a prodrug compound described above wherein G⁴ and G⁵ are independently H, or OH and not a prodrug moiety and at least one of G⁴ and G⁵ is OH and not a prodrug moiety.

In one aspect of the present invention, there is provided a prodrug compound described above wherein the prodrug moiety is an ester prodrug moiety.

In one aspect of the present invention, there is provided a prodrug compound described above wherein the prodrug moiety is a 1 to 6 carbon alkyl-ester moiety.

In one aspect of the present invention, there is provided a prodrug compound described above wherein the prodrug moiety is not a 1 to 6 carbon alkyl-ester moiety.

In one aspect of the present invention, there is provided a prodrug compound described above for use in the treatment of hepatitis C.

In one aspect of the present invention, there is provided use of the prodrug compound described above for the treatment of hepatitis C.

In one aspect of the present invention, there is provided use of the prodrug compound described above for the preparation of a medicament for the treatment of hepatitis C.

In one aspect of the present invention, there is provided use of a compound of Formula 5:

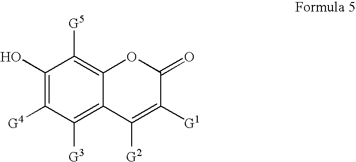

wherein G¹ is H or OH; G² is H, R′, or CO₂R; G³, G⁴ and G⁵ are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂; and R′ is a one to four carbon alkyl group, for treatment of hepatitis C. The use may be for preparation of a medicament.

In one aspect of the present invention, there is provided a use described above wherein G¹ is OH.

In one aspect of the present invention, there is provided a use described above wherein R′ is selected from the group consisting of: methyl and ethyl.

In one aspect of the present invention, there is provided a use described above wherein G² is selected from the group consisting of: H, Me, Et, CO₂Me, and CO₂Et.

In one aspect of the present invention, there is provided a use described above wherein G³ is H.

In one aspect of the present invention, there is provided a use described above wherein G⁴ and G⁵ are independently H or OH, and at least one of G⁴ and G⁵ is OH.

In one aspect of the present invention, there is provided use of a compound having the structure:

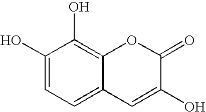

for the treatment of hepatitis C. The use may be for preparation of a medicament.

In one aspect of the present invention, there is provided use of a compound having the structure:

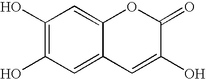

for the treatment of hepatitis C. The use may be for preparation of a medicament.

In one aspect of the present invention, there is provided use of a compound having the structure:

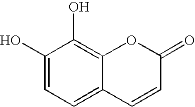

for the treatment of hepatitis C. The use may be for preparation of a medicament.

In one aspect of the present invention, there is provided use of a compound having the structure:

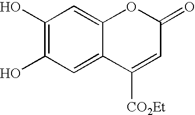

for the treatment of hepatitis C. The use may be for preparation of a medicament.

In one aspect of the present invention, there is provided use of a compound having the structure:

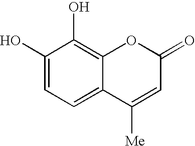

for the treatment of hepatitis C. The use may be for preparation of a medicament.

In one aspect of the present invention, there is provided use of a compound having the structure:

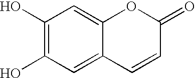

for the treatment of hepatitis C. The use may be for preparation of a medicament.

In one aspect of the present invention, there is provided use of a compound having the structure:

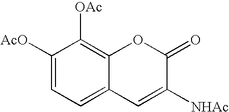

for the treatment of hepatitis C. The use may be for preparation of a medicament.

In one aspect of the present invention, there is provided a use described above wherein any one or more OH moieties is replaced with a prodrug moiety.

In one aspect of the present invention, there is provided a use described above wherein the prodrug moiety is an ester prodrug moiety.

In one aspect of the present invention, there is provided a use described above wherein the prodrug moiety is a 1 to 6 carbon alkyl-ester moiety.

In one aspect of the present invention, there is provided a use described above wherein the prodrug moiety is not a 1 to 6 carbon alkyl-ester moiety.

In one aspect of the present invention, there is provided a method for treating hepatitis C comprising administering a therapeutically effective amount of a compound of Formula 5 or a prodrug compound of Formula 5:

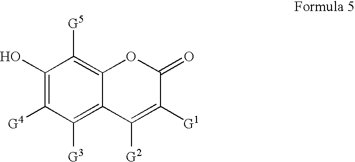

wherein G¹ is H, or OH; G² is H, R′, or CO₂R′; G³, G⁴ and G⁵, are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂; R′ is a one to four carbon alkyl group; and from 0 to 5 OH moieties are replaced with a prodrug moiety, to a subject in need thereof.

In one aspect of the present invention, there is provided a method described above wherein G¹ is OH or a prodrug moiety.

In one aspect of the present invention, there is provided a method described above wherein G¹ is OH and not a prodrug moiety.

In one aspect of the present invention, there is provided a method described above wherein R′ is selected from the group consisting of: methyl and ethyl.

In one aspect of the present invention, there is provided a method described above wherein G² is selected from the group consisting of: H, Me, Et, CO₂Me, and CO₂Et.

In one aspect of the present invention, there is provided a method described above wherein G³ is H.

In one aspect of the present invention, there is provided a method described above wherein G⁴ and G⁵ are independently H, OH or a prodrug moiety and at least one of G⁴ and G⁵ is OH or a prodrug moiety.

In one aspect of the present invention, there is provided a method described above wherein G⁴ and G⁵ are independently H or OH and not a prodrug moiety and at least one of G⁴ and G⁵ is OH and not a prodrug moiety.

In one aspect of the present invention, there is provided a method described above wherein the prodrug moiety is an ester prodrug moiety.

In one aspect of the present invention, there is provided a method described above wherein the prodrug moiety is a 1 to 6 carbon alkyl-ester moiety.

In one aspect of the present invention, there is provided a method described above wherein the prodrug moiety is not a 1 to 6 carbon alkyl-ester moiety.

In one aspect of the present invention, there is provided a method for treating hepatitis C comprising administering a therapeutically effective amount of a compound having the structure:

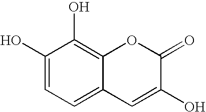

to a subject in need thereof.

In one aspect of the present invention, there is provided a method for treating hepatitis C comprising administering a therapeutically effective amount of a compound having the structure:

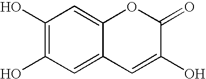

to a subject in need thereof.

In one aspect of the present invention, there is provided a method for treating hepatitis C comprising administering a therapeutically effective amount of a compound having the structure:

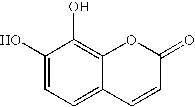

to a subject in need thereof.

In one aspect of the present invention, there is provided a method for treating hepatitis C comprising administering a therapeutically effective amount of a compound having the structure:

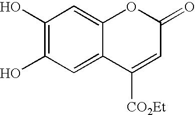

to a subject in need thereof.

In one aspect of the present invention, there is provided a method for treating hepatitis C comprising administering a therapeutically effective amount of a compound having the structure:

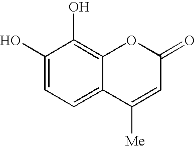

to a subject in need thereof.

In one aspect of the present invention, there is provided a method for treating hepatitis C comprising administering a therapeutically effective amount of a compound having the structure:

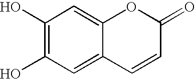

to a subject in need thereof.

In one aspect of the present invention, there is provided a method for treating hepatitis C comprising administering a therapeutically effective amount of a compound having the structure:

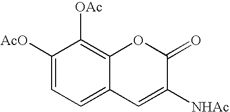

to a subject in need thereof.

In one aspect of the present invention, there is provided a method described above wherein any one or more OH moieties is replaced with a prodrug moiety.

In one aspect of the present invention, there is provided a method described above wherein the prodrug moiety is an ester prodrug moiety.

In one aspect of the present invention, there is provided a method described above wherein the prodrug moiety is a 1 to 6 carbon alkyl-ester moiety.

In one aspect of the present invention, there is provided a method described above wherein the prodrug moiety is not a 1 to 6 carbon alkyl-ester moiety.

### BRIEF DESCRIPTION OF DRAWINGS

FIG. 1: Pictorial representation of the high-throughput screening in vitro FRET-based assay system for monitoring HCV NS3/4A protease activity using BSIQFS1.

FIG. 2: Determination of ID₅₀ values for coumarin-like small molecules.

FIG. 3: Determination of mechanism of inhibition of 3,6,7-trihydroxycoumarin using Lineweaver-Burk plot (non-competitive).

FIG. 4: Residual Activity vs. Concentration of inhibitor for C21 and C11.

FIG. 5: Dose-response curve for compounds C11, C12, C21, C22 and C10 against HCV NS3^{PRO}.

FIG. 6: Dose-response curve for compounds C11, C12, C21, C22 and C10 against SARS 3CL^{PRO}.

### DETAILED DESCRIPTION

Any terms not directly defined herein shall be understood to have the meanings commonly associated with them as understood within the art of the invention. Certain terms are discussed below, or elsewhere in the specification, to provide additional guidance to the practitioner in describing the devices, methods and the like of embodiments of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments of the invention herein.

In some embodiments, the invention provides a method of treating hepatitis C through the administration to a patient of at least one substituted coumarin compound, structural analogues of such compounds, their pharmaceutically-acceptable salts or other pharmaceutically-acceptable compositions, comprising at least one of the substituted coumarin compounds described herein, or its salt, as a prodrug, precursor to, or parent compound of, an inhibitor of hepatitis C virus NS3/NS4A protease.

In one embodiment this invention includes the therapeutic use of a compound of either Formula 1 or Formula 2 for the treatment of hepatitis C.

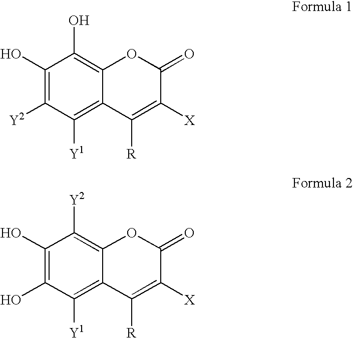

wherein, X is either H or OH; and, R is either H, R′, or CO₂R′. Y¹ and Y² are independently selected from the group: H, R′, OH, OR′, F, Cl, Br, I, NH₂, NHR′, NR′₂, CN, SH, SR′, SO₃H, SO₃R′, SO₂R′, OSO₃R′, and NO₂. Wherein R′ is a one to four carbon alkyl group, preferably methyl or ethyl.

In one embodiment this invention includes the therapeutic use of a compound of either Formula 3 or Formula 4 for the treatment of hepatitis C.

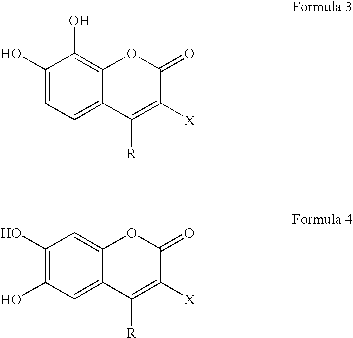

wherein, X is either H or OH; and, R is either H, R′, or CO₂R′ (where R′ is a one to four carbon alkyl group, preferably methyl or ethyl.)

Combining Formulas 1, 2, 3 and 4 provides Formula 5:

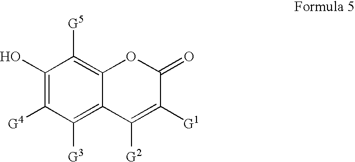

wherein G¹ is either H or OH; G² is either H, R′, or CO₂R′; and G³, G⁴ and G⁵, are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂, wherein R′ is a one to four carbon alkyl group. In some embodiments of Formula 5, R′ is selected from the group consisting of: methyl and ethyl. In some embodiments of Formula 5, G² is selected from the group consisting of: H, Me, Et, CO₂Me, and CO₂Et. In some embodiments of Formula 5, G³ is H. In some embodiments of Formula 5, G⁴ and G⁵ are independently H or OH, provided that at least one of G⁴ and G⁵ is OH.

In one embodiment this invention includes the therapeutic use of a compound of Formula 5 for the treatment of hepatitis C.

In another aspect the invention is the therapeutic use of a compound listed in Table 1 (below) for the treatment of hepatitis C.

TABLE 1

| | | ID₅₀ (μm)-by |
|---|---|---|
| Number | Compound | kinetic assay |
| C21 (AKA 6 from synthetic scheme) | 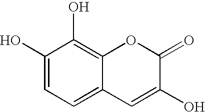 | 1.6 |
| C11 (AKA 5 from synthetic scheme below) | 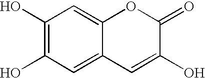 | 3.07 |
| C10 | 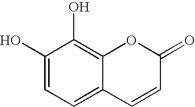 | 25 |
| MC8 | 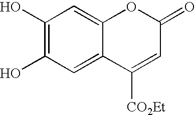 | 25.2 |
| C8 | 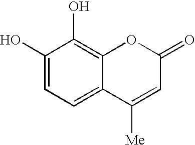 | 108 |
| C4 | 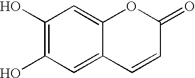 | 279.8 |

In some embodiments, the invention comprises a prodrug of a compound according to any one of Formulas 1, 2, 3, 4 or 5 or a prodrug of a compound set out in Table 1. A prodrug of a compound according to any one of Formulas 1, 2, 3, 4 or 5 or a prodrug of a compound set out in Table 1 is a compound according to any one of Formulas 1, 2, 3, 4 or 5 or a compound set out in Table 1 wherein an OH moiety is replaced with a prodrug moiety.

A prodrug moiety is a moiety that replaces an OH moiety on a core, to form a prodrug compound of this invention. The core is compound according to any one of Formulas 1, 2, 3, 4 or 5 or a compound set out in Table 1. In some embodiments, prodrug moieties may be moieties that may be cleaved in vivo such that a compound of the core is produced via cleavage of the prodrug moiety thereby separating the prodrug moiety from the core. In some embodiments, the prodrug moiety may be cleaved enzymatically. In some embodiments, in vivo cleavage of the prodrug moiety from the core results in the formation of a core comprising an OH moiety where the prodrug moiety was bonded to the core prior to cleavage. Prodrug moieties comprising an ester moiety may provide formation of a core comprising an OH moiety where the ester prodrug moiety was bonded to the core prior to cleavage. Non-limiting examples of prodrug moieties include 1 to 6 carbon alkyl-esters. Such 1 to 6 carbon alkyl-esters are considered to be ester prodrug moieties. Specific, non-limiting examples of prodrug moieties are described below in Tables 2 and 3. Table 3 sets out specific ester prodrug moieties. In the following Tables 2 and 3, each R is independently a linear, branched, or cyclic, saturated or unsaturated one to ten carbon alkyl group that is unsubstituted or is substituted with one or more of: OH, ═O, SH, F, Br, Cl, I, NH₂, —NHR′, —NR′₂, NO₂, —CO₂H, —CO₂R′, and epoxide; and R′ is a linear, branched, or cyclic, saturated or unsaturated one to ten carbon alkyl group that is unsubstituted or substituted with one or more of: OH, ═O, SH, F, Br, Cl, I, NH₂, —NHR″, —-NR″₂, NO₂ and —CO₂H where R″ is a linear, branched, or cyclic, saturated or unsaturated one to ten carbon alkyl group.

TABLE 2
PRODRUG MOIETIES

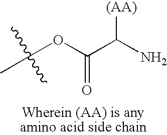

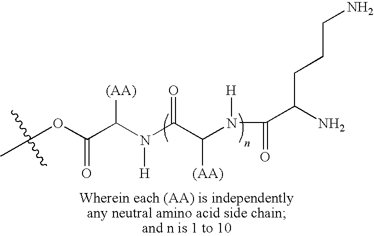

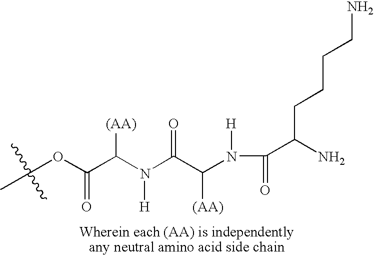

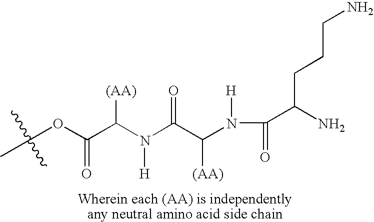

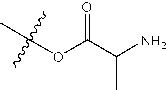

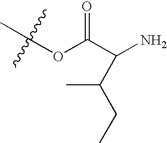

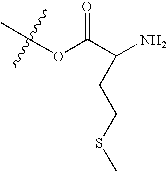

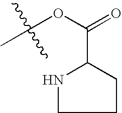

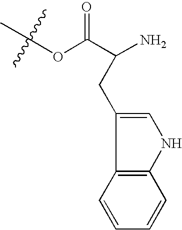

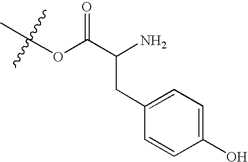

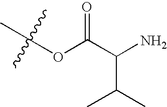

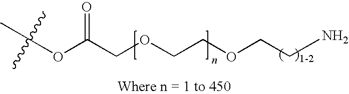

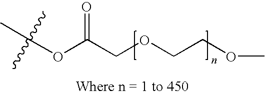

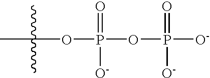

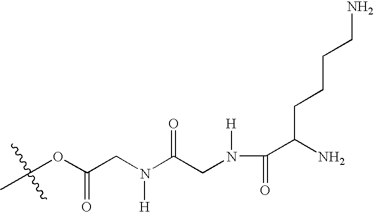

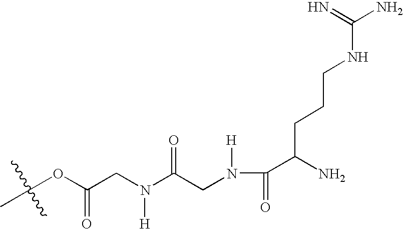

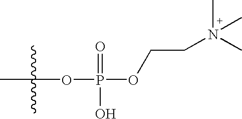

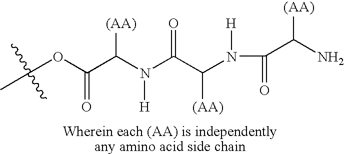

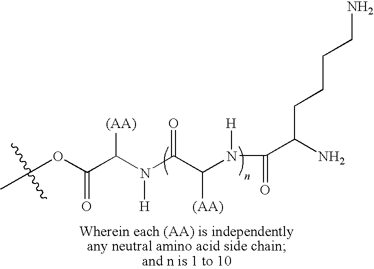

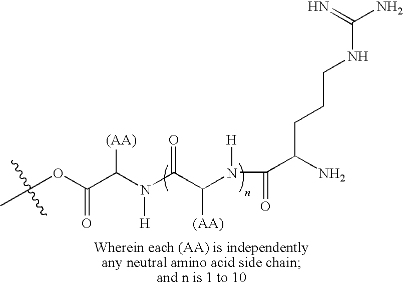

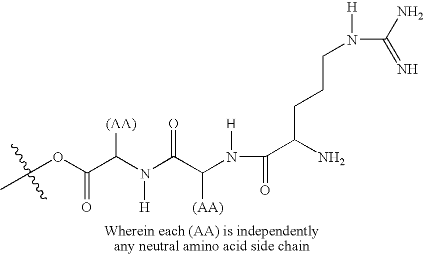

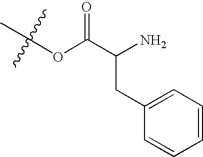

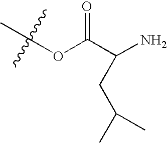

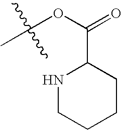

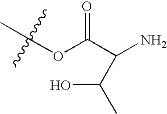

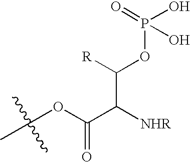

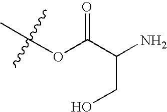

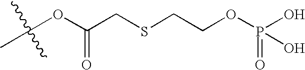

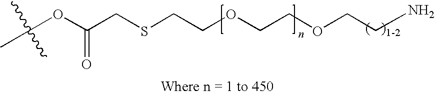

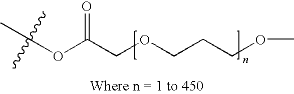

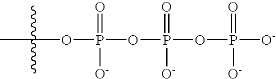

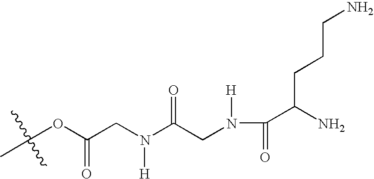

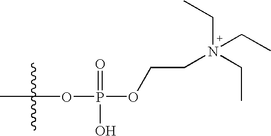

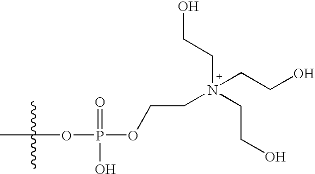

In some particular embodiments, each R as set out in Table 2 may be independently selected from H, methyl or acyl.

TABLE 3
ESTER PRODRUG MOIETIES

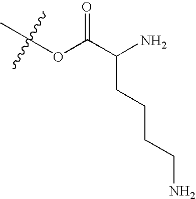

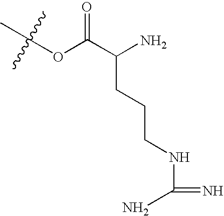

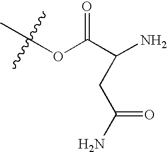

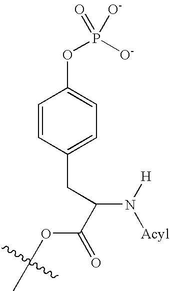

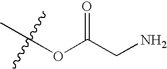

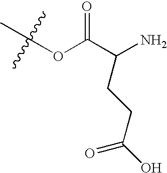

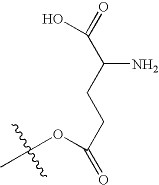

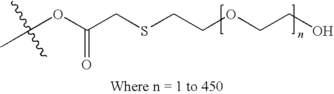

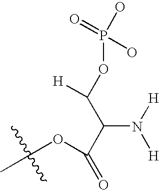

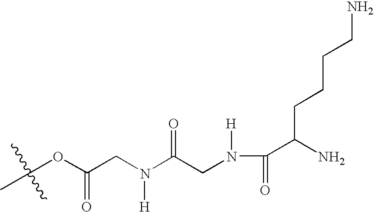

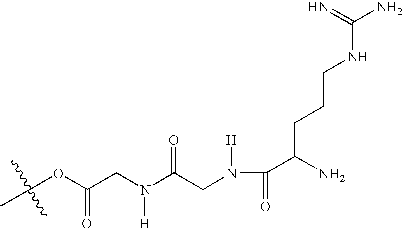

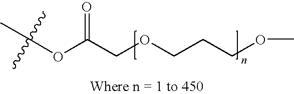

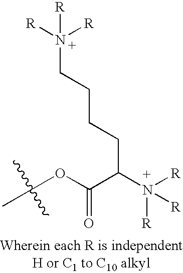

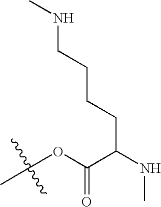

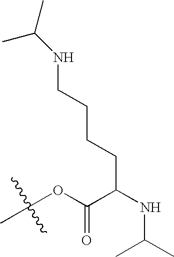

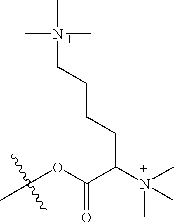

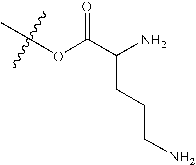

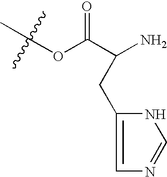

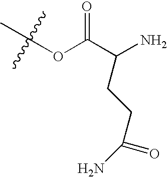

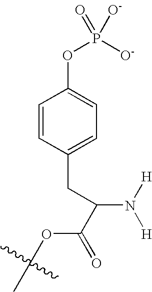

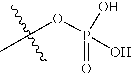

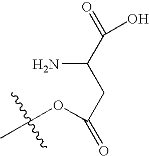

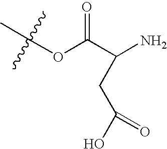

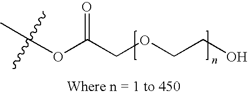

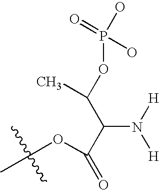

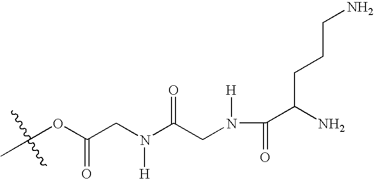

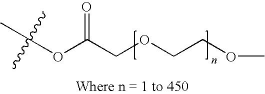

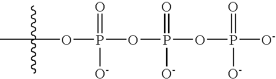

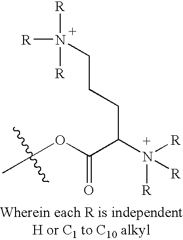

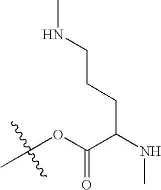

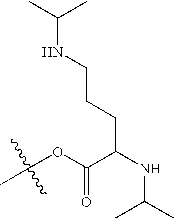

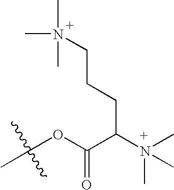

In one embodiment this invention includes a prodrug compound of Formula 5:

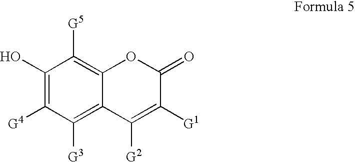

wherein G¹, G², G³, G⁴ and G⁵ are as described above for Formula 5 and one or more OH moieties are replaced with a prodrug moiety. In some embodiments the prodrug moiety is a substituent on the carbons at positions 3, 5, 6, 7 and/or 8. In some embodiments the prodrug moiety is an ester prodrug moiety as described above. In some embodiments the prodrug moiety is a 1 to 6 carbon alkyl-ester. In some embodiments the prodrug moiety is as described above, but not a 1 to 6 carbon alkyl-ester moiety.

In one embodiment this invention includes the therapeutic use of a prodrug compound of Formula 5 for the treatment of hepatitis C.

Compositions according to some embodiments of the invention may be formulated by means known in the art. For example, a composition suitable for parenteral administration may be dissolved in sterile water or saline, or other sterile aqueous media. Compositions suitable for enteric administration may be provided in a liquid, tablet, suspension or gel form. The composition may be formulated for timed or sustained release. Compositions suitable for topical administration may be provided as an ointment, cream, gel, liquid, powder, patch or the like. The composition for topical application may further be formulated for timed or sustained release. Various techniques are known to those of skill in the art of formulating pharmaceutical compositions, and may be found in, for example, Remington: the Science & Practice of Pharmacy by Alfonso Gennaro, 20th ed., Williams & Wilkins, (2000).

Compositions in accordance with this invention or for use in this invention may be administered to a patient by standard procedures, including topical, oral, inhalation, intramuscular, intravenous, or intraperitoneal administration. Dosage and duration of treatment may be determined by a practitioner in accordance with standard protocols and information concerning a chosen composition.

Compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

Many compounds of this invention or for use in this invention are generally water soluble and may be formed as salts. In such cases, pharmaceutical compositions in accordance with this invention may comprise a salt of such a compound, preferably a physiologically acceptable salt, which are known in the art. Pharmaceutical preparations will typically comprise one or more carriers acceptable for the mode of administration of the preparation, be it by injection, inhalation, topical administration, lavage, or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as improved liver function or reduced virus count. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as liver function or reduced virus count. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

As used herein, a “subject” may be a human, non-human primate, rat, mouse, cow, horse, pig, sheep, goat, dog, cat, etc. The subject may be suspected of having or at risk for having hepatitis C. Diagnostic methods for hepatitis C and the clinical delineation of hepatitis C diagnoses are known to those of ordinary skill in the art.

Various alternative embodiments and examples of the invention are described herein. These embodiments and examples are illustrative and should not be construed as limiting the scope of the invention.

### Example 1

### Synthesis of Trihydroxylated Coumarins

3,6,7- and 3,7,8-trihydroxycoumarins were synthesised from the corresponding trihydroxybenzaldehyde using the method of Cotelle and co-workers, as shown below. (Cotelle, P. and Vezin, H., (2003) Res. Chem. Intermed., 29, 365-377; Bailly, F., Maurin, C., Teissier, E., Vezin, H and Cotelle, P. (2004) Bioorg. Med. Chem. 12, 5611-5618)

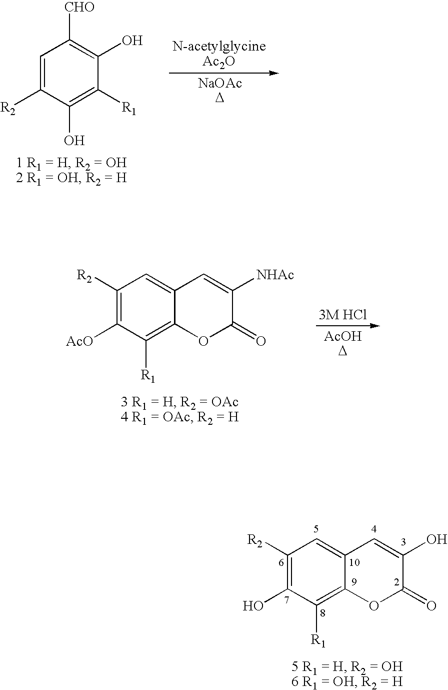

### Example 1A

### Synthesis of 3,6,7-trihydroxycoumarin

2,4,5-trihydroxybenzaldehyde (1) (1.54 g, 9.96 mmol), N-acetylglycine (1.89 g, 16.14 mmol, 1.62 equiv) and NaOAc (1.30 g, 15.87 mmol, 1.59 equiv) were refluxed in Ac₂O (10 mL) for 1 hr. The resulting mixture was cooled to room temperature and iced H₂O (10 mL) was added, causing precipitation. This suspension was placed in a freezer for 15 mins and was then filtered. The precipitate was washed with copious amounts of H₂O and 50% v/v MeOH_{(aq)} to give 3-acetamido-6,7-diacetoxycoumarin (3) (1.22 g, 3.82 mmol, 38%).

3-Acetamido-6,7-diacetoxycoumarin (3): Yellow solid; ¹H NMR (600 MHz, CDCl₃) δ_{H}: 8.64 (s, 1H), 8.06 (s, 1H), 7.35 (s, 1H), 7.24 (s, 1H), 2.33 (s, 3H), 2.33 (s, 3H), 2.11 (s, 3H); ¹³C NMR (150 MHz, CDCl₃) δ_{C}: 169.4, 168.1, 167.7, 158.2, 147.2, 142.9, 139.4, 124.1, 122.0, 121.4, 118.1, 111.8, 24.7, 20.6, 20.6; 3-Acetamido-6,7-diacetoxycoumarin (3) (1.20 g, 3.77 mmol) was refluxed in 3 M HCl (50 mL) and AcOH (2 mL) for 1 hr. The reaction mixture was cooled to room temperature and extracted with EtOAc (3×75 mL). The organic layer was dried and evaporated and then recrystalised from H₂O to give 3,6,7-trihydroxycoumarin (5) (0.10 g, 0.52 mmol, 14%).

3,6,7-Trihydroxycoumarin (5): Red solid; ¹H NMR (600 MHz, DMSO) δ_{H}: 9.68 (s, 2H), 9.18 (s, 1H), 6.99 (s, 1H), 6.82 (s, 1H), 6.71 (s, 1H); ¹³C NMR (150 MHz, DMSO) δ_{C}: 158.9, 146.5, 143.3, 143.1, 138.8, 116.3, 111.9, 110.8, 102.5; HRESIMS (m/z): Calc'd for C₉H₆O₅ 217.0113, found 217.0112 [M+Na]⁺, Δ−0.4 ppm.

### Example 1B

### Synthesis of 3,7,8-trihydroxycoumarin

2,3,4-trihydroxybenzaldehyde (2) (0.98 g, 6.33 mmol), N-acetylglycine (1.21, 10.34 mmol, 1.63 equiv) and NaOAc (0.82 g, 9.96 mmol, 1.57 equiv) were refluxed in Ac₂O (7.5 mL) for 1 hr. The resulting mixture was cooled to room temperature and iced H₂O (10 mL) was added, causing an oil to form. The reaction mixture was extracted with EtOAc (3×50 mL) which was washed with H₂O (20 mL), saturated NaHCO₃ (3×30 mL) and H₂O (3×30 mL). The organic layer was evaporated and the resulting solid recrystalised from 50% v/v MeOH_{(aq)} to give 3-acetamido-7,8-diacetoxycoumarin (4) (1.10 g, 3.45 mmol, 54%).

3-Acetamido-7,8-diacetoxycoumarin (4): Yellow solid; ¹H NMR (400 MHz, CDCl₃) δ_{H}: 8.67 (s, 1H), 8.04 (s, 1H), 7.39 (d, 8.7, 1H), 7.15 (d, 8.7, 1H), 2.42 (s, 3H), 2.33 (s, 3H), 2.24 (s, 3H); ¹³C NMR (100 MHz, CDCl₃) δ_{C}: 169.5, 167.9, 167.2, 157.4, 143.4, 142.6, 130.0, 124.6, 123.8, 122.5, 119.8, 118.7, 24.6, 20.6, 20.2; HRESIMS (m/z): Calc'd for C₁₅H₁₄NO₇ 320.0770, found 320.0765 [M+H]⁺, Δ−1.6 ppm.

3-Acetamido-7,8-diacetoxycoumarin (4) (0.51 g, 1.60 mmol) was refluxed in 3 M HCl (25 mL) and AcOH (1 mL) for 1 hr. The reaction mixture was cooled to room temperature and extracted with EtOAc (3×50 mL). The organic layer was evaporated and then recrystalised from isopropyl alcohol to give 3,7,8-trihydroxycoumarin (6) (0.074 g, 0.38 mmol, 24%) as the mother liquor.

3,7,8-Trihydroxycoumarin (6): Red solid; ¹H NMR (600 MHz, DMSO) δ_{H}: 9.77 (s, 1H), 9.63 (s, 1H), 9.21 (s, 1H), 7.02 (s, 1H), 6.81 (d, 8.5, 1H), 6.74 (d, 8.5, 1H); ¹³C NMR (150 MHz, DMSO) δ_{C}: 158.7, 146.1, 139.4, 138.4, 132.1, 116.7, 116.2, 113.2, 112.8; HRESIMS (m/z): Calc'd for C₉H₆O₅ 217.0113, found 217.0110 [M+Na]⁺, Δ−1.4 ppm.

### Example 2

### Activity of the Compounds

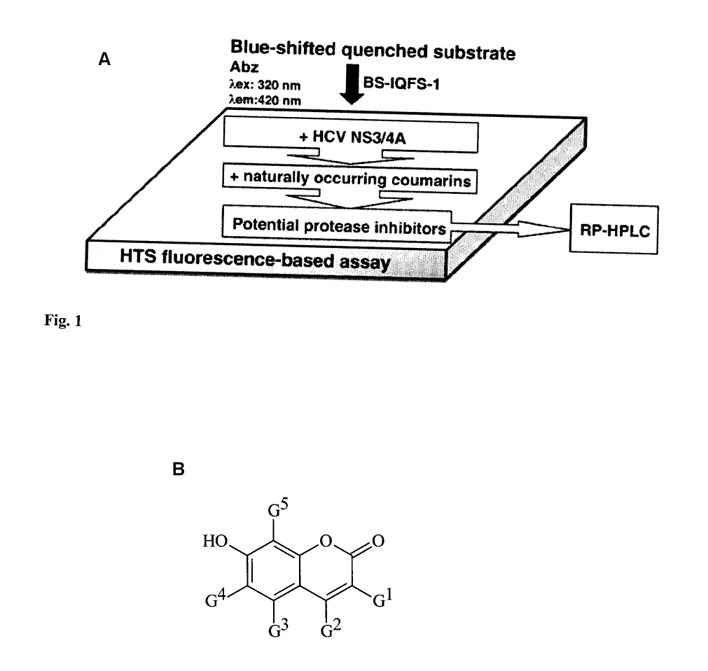

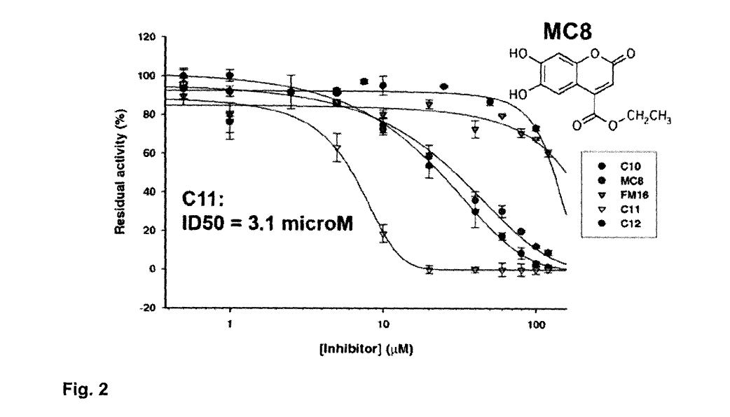

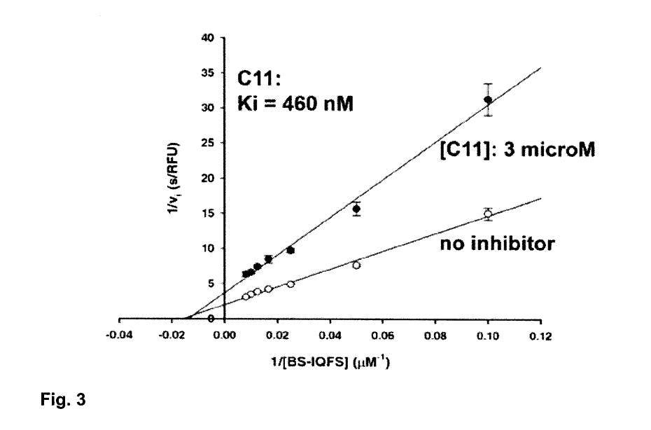

The compounds that are the subject of this invention were discovered using our high-throughput FRET-protease assays. The protease assays are based on specific and sensitive internally quenched fluorogenic substrates (Methods used are fully described in Hamill P. & Jean F (2005) Biochemistry 44: 6586-6596; and, Richer M. et al (2004) JBC 279: 10222-10227, which are incorporated herein by reference)—see FIG. 1. Of the inhibitory compounds identified, it was determined that best lead compounds with anti-HCV activity are Compound C11 (3,6,7-trihydroxycoumarin; ID₅₀=3.0 μM; Ki=460 nM) and C21 (3,7,8-trihydroxycoumarin; ID₅₀=1.6 μM). Furthermore, two compounds were identified with ID₅₀ values in the low micromolar range, C10 (ID₅₀: 23.8 μM) and MC8 (ID₅₀: 28.6 μM)—see FIG. 2. The two best coumarin derivatives (C11 and C21) act as a non-competitive protease inhibitors (see FIG. 3) and are known to have antioxidant properties, which will prove an additional asset in countering the oxidative stress induced by HCV infection.

### Analysis of Kinetic Parameters

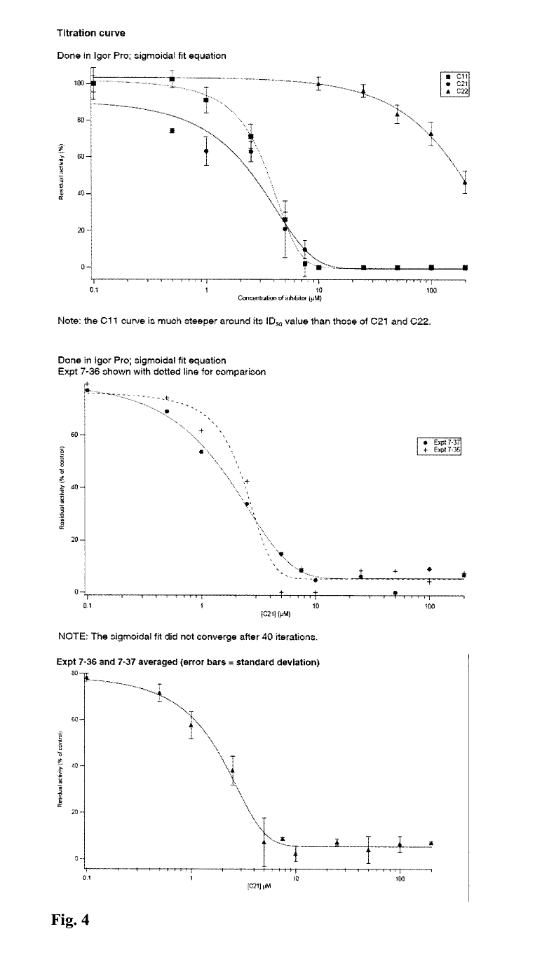

See also FIG. 4

Taken from Igor analysis:

| Expt | Compound | k₀ (base) | k₁ (max) | k₂ (x_{half}) | k₃ (rate) | $\frac{1}{2} {y}_{max}$ | $\frac{1}{10} {y}_{max}$ |
|---|---|---|---|---|---|---|---|
| 7-36 | C21 | 79.759 | −74.5116 | 2.33304 | 0.772954 | 50 | 10 |
| 7-37 | C21 | 285.386 | −280.039 | −2.12861 | 2.08466 | 50 | 10 |
| Avg | C21 | 107.014 | −101.545 | 1.27812 | 1.33719 | 50 | 10 |

NOTES
All values based on HPLC calculated area under the “Cleaved Substrate” peak.
0.1 μM reading used as 100% control.
The sigmoidal fit algorithm for Expt 7-37 did not converge after 40 iterations.
Sigmoidal fit equation (used in Igor):
$y = base + \frac{max}{1 + {e}^{\frac{{x}_{half} - x}{rate}}} or y = {k}_{0} + \frac{{k}_{1}}{1 + {e}^{\frac{- ( x - {k}_{2} )}{{k}_{3}}}}$
solved for x:
$x = {k}_{2} - {k}_{3} ln ( \frac{{k}_{1}}{y - {k}_{0}} - 1 )$
${ID}_{50} value is the x value where y = \frac{1}{2} {y}_{max}$
${ID}_{90} value is the x value where y = \frac{1}{10} {y}_{max}$

| | | |
|---|---|---|
| Expt 7-36: | ID₅₀ = 2.02 μM | ID₉₀ = 4.41 μM |
| Expt 7-37: | ID₅₀ = 1.34 μM | ID₉₀ = 6.38 μM |
| Average: | ID₅₀ = 1.61 ± 0.48 μM | ID₉₀ = 4.41 ± 1.39 μM |

The following Table 4 sets out the results of the compounds tested and analysed.

TABLE 4

| | | | ID50 (μM) |
|---|---|---|---|
| No. | Full Name | Structure | (by kinetic assay) |
| C11 | 3-hydroxyesculetin | 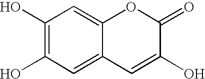 | 3.07 |
| C12 | 3-aminoacyl-6,7- diacetoxycoumarin | 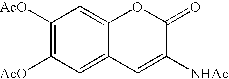 | >300 |
| C13 | Umbelliferone | 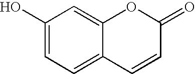 | >300 |
| C1 | 7-hydroxycoumarin-4-ethyl carboxylate | 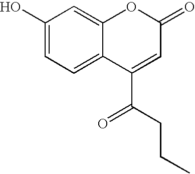 | >300 |
| C14 | 3-hydroxycoumarin | 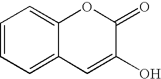 | >300 |
| C15 | 4-hydroxycoumarin | 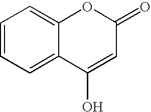 | >300 |
| C8 | 4-methyldaphnetin | 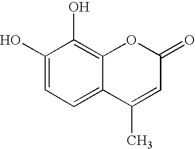 | 108.8 |
| C10 | Daphnetin | 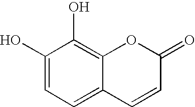 | 25.0 |
| C16 | 7,8-dihydroxy-4- methylcoumarin-3-acetic acid | 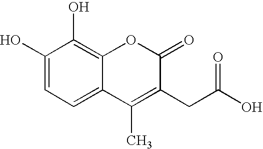 | >300 |
| C17 | 7-methoxydaphnetin | 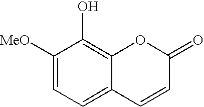 | >300 |
| C18 | 7,8-dimethoxydaphnetin | 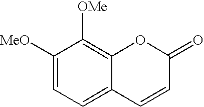 | >300 |
| C19 | Fraxetin | 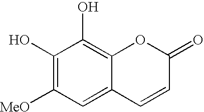 | >300 |
| C9 | 5,7-dihydroxy-4- methylcoumarin | 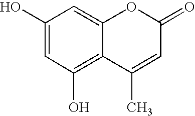 | >300 |
| C4 | Esculetin | 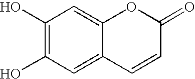 | 279.8 |
| MC8 | Esculetin-4-ethyl ester | 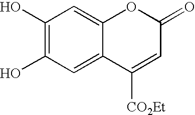 | 25.2 |
| C2 | 4-carboxyesculetin | 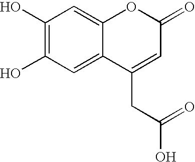 | >300 |
| C3 | 4-methylesculetin | 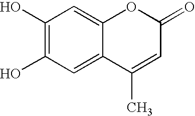 | >300 |
| C20 | Nordalbergin | 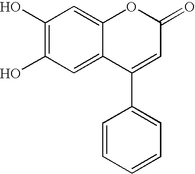 | >300 |
| FM373 | 7,8-dihydroxycoumarin-4- acetic acid | 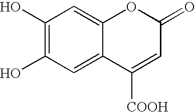 | >300 |
| C5 | Scoparone | 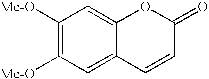 | >300 |
| C6 | Isoscopoletin | 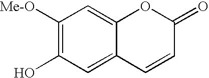 | >300 |
| C7 | Scopoletin | 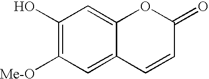 | >300 |
| FM16 | Scopoletin-4-ethyl ester | 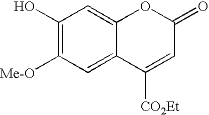 | 393 |
| FM370 | 7-hydroxy-6- methyoxycoumarin-4- acetic acid | 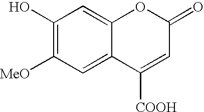 | >300 |
| C21 | 3,7,8-trihydroxycoumarin | 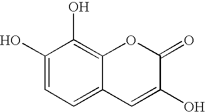 | 2.1 |
| C22 | 3-aminoacyl-7,8- diacetoxycoumarin | 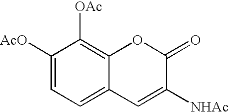 | 185 |

### Example 3

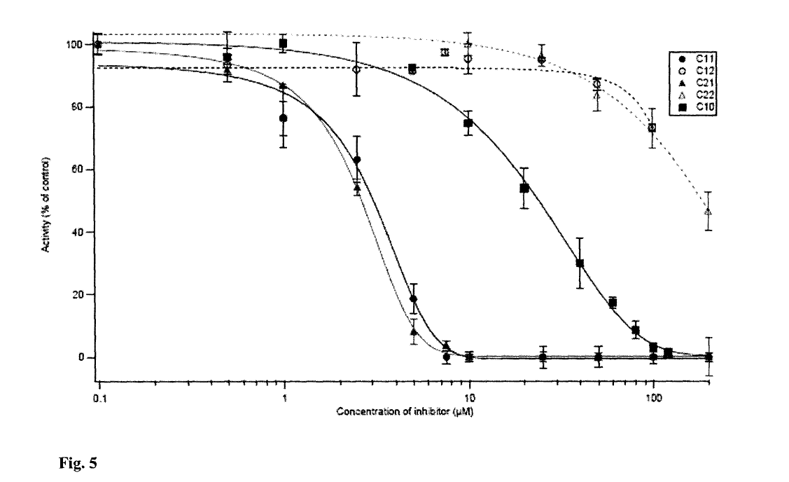

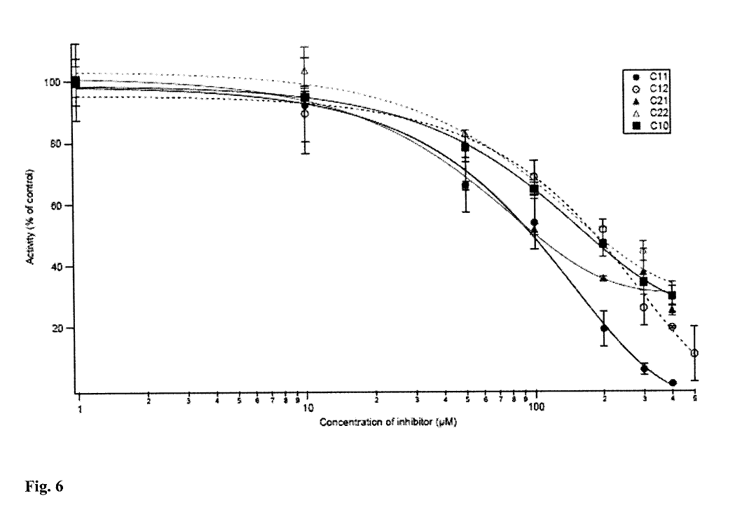

Compounds C11, C12, C21, C22 and C10 were tested as described above in Example 2 (methods used are fully described in Hamill P. & Jean F (2005) Biochemistry 44: 6586-6596; and, Richer M. et al (2004) JBC 279: 10222-10227, which are incorporated herein by reference) and tested in a similar assay for SARS-coronavirus (methods used are fully described in Hamill et al. (2006) Biol. Chem., vol. 387, pp. 1063-1074, incorporated herein by reference) and the results were compared. The results are illustrated in FIGS. 5 and 6 and set out in Table 5 below. The SARS 3CL results indicate no discrepancies in activity between C11 (ID50: 97 μM) and C12 (ID50: 183 μM) or C21 (100 μM) and C22 (ID50: 184 μM). The value for C10 is in the same range (ID50: 168 μM). The compounds are inhibitory within a similar range +/±2 fold with respect to SARS. This is in contrast to the results observed for HCV NS3/4A: at least 100 fold difference between C12/C22 and C11/C21. This suggests the selectivity of C11 and C21 for HCV when tested against two different classes of viral proteases: HCV NS3/4A and SARS-3CL protease.

TABLE 5

| | | | ID50 (μM) | |
|---|---|---|---|---|
| | | | (by kinetic assay) | |
| No. | Full Name | Structure | HCV NS3^{PRO} | SARS 3CL^{PRO} |
| C11 | 3-hydroxyesculetin | 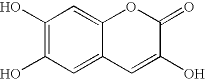 | 3.07 | 96.6 |
| C12 | 3-aminoacyl-6,7- diacetoxycoumarin | 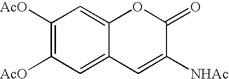 | >300 | 183 |
| C21 | 3,7,8- trihydroxycoumarin | 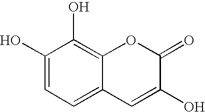 | 2.1 | 100 |
| C22 | 3-aminoacyl-7,8- diacetoxycoumarin | 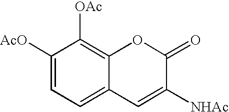 | 185 | 184 |
| C10 | Daphnetin | 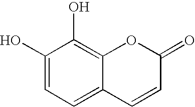 | 25.0 | 168 |

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

What is claimed is:

1. A prodrug compound of Formula 5: 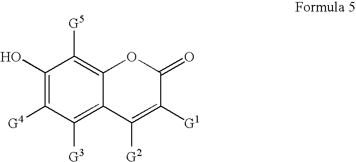
wherein
G¹ is H or OH;
G² is H, R′, or CO₂R′;
G³, G⁴ and G⁵, are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂;
each R′ is independently a one to four carbon alkyl group; and
from 1 to 5 OH moieties are replaced with a prodrug moiety.

2. The prodrug compound of claim 1 wherein G¹ is OH or a prodrug moiety.

3. The prodrug compound of claim 1 or 2 wherein G¹ is OH and not a prodrug moiety.

4. The prodrug compound of any one of claims 1 to 3 wherein each R′ is independently selected from the group consisting of: methyl and ethyl.

5. The prodrug compound of any one of claims 1 to 4 wherein G² is selected from the group consisting of: H, Me, Et, CO₂Me, and CO₂Et.

6. The prodrug compound of any one of claims 1 to 5 wherein G³ is H.

7. The prodrug compound of any one of claims 1 to 6 wherein G⁴ and G⁵ are independently H, OH or a prodrug moiety and at least one of G⁴ and G⁵ is OH or a prodrug moiety.

8. The prodrug compound of any one of claims 1 to 7 wherein G⁴ and G⁵ are independently H, or OH and not a prodrug moiety and at least one of G⁴ and G⁵ is OH and not a prodrug moiety.

9. The prodrug compound of any one of claims 1 to 8 wherein the prodrug moiety is an ester prodrug moiety.

10. The prodrug compound of any one of claims 1 to 8 wherein the prodrug moiety is a 1 to 6 carbon alkyl-ester moiety.

11. The prodrug compound of any one of claims 1 to 8 wherein the prodrug moiety is not a 1 to 6 carbon alkyl-ester moiety.

12. The prodrug compound of any one of claims 1 to 11 for use in the treatment of hepatitis C.

13. Use of the prodrug compound of any one of claims 1 to 11 for the treatment of hepatitis C.

14. Use of the prodrug compound of any one of claims 1 to 11 for the preparation of a medicament for the treatment of hepatitis C.

15. Use of a compound of Formula 5: 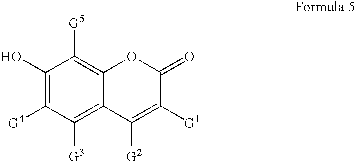
wherein
G¹ is H or OH;
G² is H, R′, or CO₂R;
G³, G⁴ and G⁵ are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂; and
R′ is a one to four carbon alkyl group,
for treatment of hepatitis C.

16. Use of a compound of Formula 5: 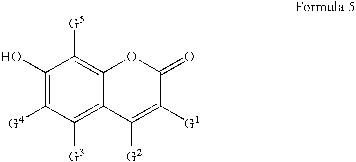
wherein
G¹ is H or OH;
G² is H, R′, or CO₂R;
G³, G⁴ and G⁵ are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂; and
R′ is a one to four carbon alkyl group,
for preparation of a medicament for treatment of hepatitis C.

17. The use of claim 15 or 16 wherein G¹ is OH.

18. The use of any one of claims 15 to 17 wherein R′ is selected from the group consisting of: methyl and ethyl.

19. The use of any one of claims 15 to 18 wherein G² is selected from the group consisting of: H, Me, Et, CO₂Me, and CO₂Et.

20. The use of any one of claims 15 to 19 wherein G³ is H.

21. The use of any one of claims 15 to 20 wherein G⁴ and G⁵ are independently H or OH, and at least one of G⁴ and G⁵ is OH.

22. Use of a compound having the structure: 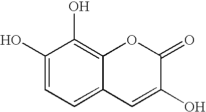
for the treatment of hepatitis C.

23. Use of a compound having the structure: 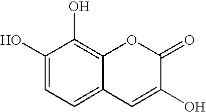
for the preparation of a medicament for the treatment of hepatitis C.

24. Use of a compound having the structure: 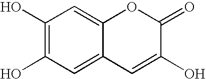
for the treatment of hepatitis C.

25. Use of a compound having the structure: 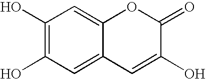
for the preparation of a medicament for the treatment of hepatitis C.

26. Use of a compound having the structure: 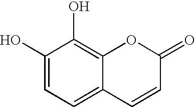
for the treatment of hepatitis C.

27. Use of a compound having the structure: 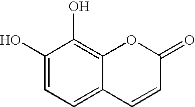
for the preparation of a medicament for the treatment of hepatitis C.

28. Use of a compound having the structure: 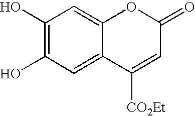
for the treatment of hepatitis C.

29. Use of a compound having the structure: 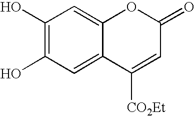
for the preparation of a medicament for the treatment of hepatitis C.

30. Use of a compound having the structure: 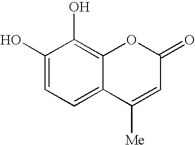
for the treatment of hepatitis C.

31. Use of a compound having the structure: 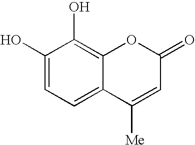
for the preparation of a medicament for the treatment of hepatitis C.

32. Use of a compound having the structure: 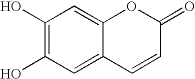
for the treatment of hepatitis C.

33. Use of a compound having the structure: 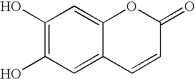
for the preparation of a medicament for the treatment of hepatitis C.

34. Use of a compound having the structure: 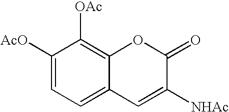
for the treatment of hepatitis C.

35. Use of a compound having the structure: 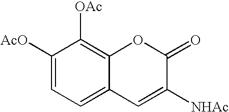
for the preparation of a medicament for the treatment of hepatitis C.

36. The use of any one of claims 22 to 35 wherein any one or more OH moieties is replaced with a prodrug moiety.

37. The use of claim 36 wherein the prodrug moiety is an ester prodrug moiety.

38. The use of claim 36 or 37 wherein the prodrug moiety is a 1 to 6 carbon alkyl-ester moiety.

39. The use of claim 36 or 37 wherein the prodrug moiety is not a 1 to 6 carbon alkyl-ester moiety.

40. A method for treating hepatitis C comprising administering a therapeutically effective amount of a compound of Formula 5 or a prodrug compound of Formula 5: 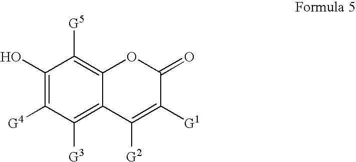
wherein
G¹ is H, or OH;
G² is H, R′, or CO₂R′;
G³, G⁴ and G⁵, are each independently selected from the group consisting of: H, R′, OH, OR′, F, Cl, Br, I, NH₂, NHR′, NR′₂, CN, SH, SR′, SO₃H, SO₃R′, SO₂R′, OSO₃R′, and NO₂;
R′ is a one to four carbon alkyl group; and
from 0 to 5 OH moieties are replaced with a prodrug moiety,
to a subject in need thereof.

41. The method of claim 40 wherein G¹ is OH or a prodrug moiety.

42. The method of claim 40 or 41 wherein G¹ is OH and not a prodrug moiety.

43. The method of any one of claims 40 to 42 wherein R′ is selected from the group consisting of: methyl and ethyl.

44. The method of any one of claims 40 to 43 wherein G² is selected from the group consisting of: H, Me, Et, CO₂Me, and CO₂Et.

45. The method of any one of claims 40 to 44 wherein G³ is H.

46. The method of any one of claims 40 to 45 wherein G⁴ and G⁵ are independently H, OH or a prodrug moiety and at least one of G⁴ and G⁵ is OH or a prodrug moiety.

47. The method of any one of claims 40 to 46 wherein G⁴ and G⁵ are independently H or OH and not a prodrug moiety and at least one of G⁴ and G⁵ is OH and not a prodrug moiety.

48. The method of any one of claims 40 to 47 wherein the prodrug moiety is an ester prodrug moiety.

49. The method of any one of claims 40 to 58 wherein the prodrug moiety is a 1 to 6 carbon alkyl-ester moiety.

50. The method of any one of claims 40 to 58 wherein the prodrug moiety is not a 1 to 6 carbon alkyl-ester moiety.

51. A method for treating hepatitis C comprising administering a therapeutically effective amount of a compound having the structure: 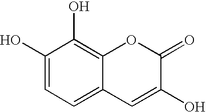
to a subject in need thereof.

52. A method for treating hepatitis C comprising administering a therapeutically effective amount of a compound having the structure: 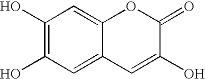
to a subject in need thereof.

53. A method for treating hepatitis C comprising administering a therapeutically effective amount of a compound having the structure: 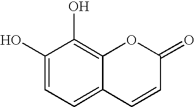
to a subject in need thereof.

54. A method for treating hepatitis C comprising administering a therapeutically effective amount of a compound having the structure: 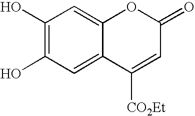
to a subject in need thereof.

55. A method for treating hepatitis C comprising administering a therapeutically effective amount of a compound having the structure: 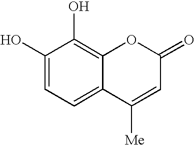
to a subject in need thereof.

56. A method for treating hepatitis C comprising administering a therapeutically effective amount of a compound having the structure: 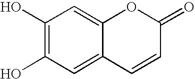
to a subject in need thereof.

57. A method for treating hepatitis C comprising administering a therapeutically effective amount of a compound having the structure: 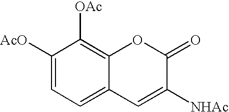
to a subject in need thereof.

58. The method of any one of claims 51 to 57 wherein any one or more OH moieties is replaced with a prodrug moiety.

59. The method of claim 58 wherein the prodrug moiety is an ester prodrug moiety.

60. The method of claim 58 or 59 wherein the prodrug moiety is a 1 to 6 carbon alkyl-ester moiety.

61. The method of claim 58 or 59 wherein the prodrug moiety is not a 1 to 6 carbon alkyl-ester moiety.
